(19) United States
(12) Patent Application Publication
(10) Pub. No.: US 2018/0280181 A1
(43) Pub. Date: Oct. 4, 2018

(54) FIXING DEVICE OF WAIST

(71) Applicant: Xieping ZHU, Shanghai (CN)

(72) Inventor: Xieping ZHU, Shanghai (CN)

(21) Appl. No.: 15/788,830

(22) Filed: Oct. 20, 2017

(30) Foreign Application Priority Data
Mar. 28, 2017 (CN) 201720311391.8

(51) Int. Cl. A61F 5/02

(52) U.S. Cl. CPC A61F 5/028

(57) ABSTRACT

A waist fixation device includes a back piece, a first elastic band, a second elastic band, and a wing piece. The first elastic band and the second elastic band are respectively arranged at the two sides of the back piece. When in use, the shape of the back piece is matched with the posterior side of the waist of the patient. The first elastic band and the second elastic band are respectively adhered on the two sides of the waist of the patient. The fixation device is fixed around the waist of the patient for supporting and fixing the waist.

### CROSS-REFERENCE TO RELATED APPLICATIONS

Pursuant to 35 U.S.C. § 119 and the Paris Convention Treaty, this application claims foreign priority to Chinese Patent Application No. 201720311391.8 filed Mar. 28, 2017, the contents of which and any intervening amendments thereto are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

### Field of the Invention

The disclosure relates to a waist fixation device. The device not only can be used for rigid fixation immediately post-fracture but can also be used following surgery.

### Description of the Related Art

When external fixation is used to treat lumbar fracture and other bone diseases, conventionally, a waistband made of canvas and leather or a gypsum tube is used. The waistband made of canvas and leather is provided with aluminum alloy strips or steel sheet strips to enhance the support to the waist. When a gypsum plaster is used for treating the fracture of the lumbar vertebra, edema of the wound may cause excessive compression of the lumbar vertebra, and thus the circulation of the blood may be affected. The waistband made of canvas and leather with reinforced support is not suitable for fixing the fracture due to the poor characteristics and the defects of the materials, and is only suitable for auxiliary treatment for lumbar muscle sprains or ordinary waist discomforts. Optionally, a prefabricated mold can also be used for fixing, but the mold is difficult to tightly attach to the body of a patient, leading to poor fixation.

### SUMMARY OF THE INVENTION

In view of the above-described problems, it is one objective of the invention to provide a waist fixation device, the device can be attached to the waist of a patient, it is convenient to use, the fixing effect is good, the fracture part is effectively supported and fixed, and the treatment and rehabilitation periods are shortened.

To achieve the above objectives, in accordance with one embodiment of the invention, there is provided a waist fixation device comprising: (A) a back piece comprising a cavity and a filling port; (B) a first elastic band comprising a first end; (C) a second elastic band comprising a second end; (D) a first fastener; and (E) a wing piece comprising a sealing cavity and a supporting bar.

The back piece is adapted to cover the posterior side of the waist of a patient. The first elastic band and the second elastic band are respectively connected to two sides of the back piece.

A first piece of the first fastener is disposed on the first end and a second piece of the first fastener is disposed on the second end to connect the first end and the second end, for keeping the fixation device wrapped around the waist. The cavity is arranged in the back piece. The filling port is arranged on the back piece and is communicated with the cavity. When in use, the polyurethane slurry is injected into the cavity through the filling port, after the polyurethane slurry enters the solid-phase softening section, a shape of the back piece is matched with a shape of the posterior side of the waist. Two wing pieces are respectively connected to outer sides of a first elastic band and a second elastic band. The sealing cavity is arranged in the wing piece, and the supporting bar is placed in the sealing cavity.

In a class of this embodiment, the waist fixation device comprises an adjustable fixation belt and a second fastener. The adjustable fixation belt is adapted to fix the fixation device on the waist. A first piece of the second fastener is disposed on the adjustable fixation belts and a second piece of the second fastener is disposed on the back piece for fixing the adjustable fixation belt on the back piece.

In a class of this embodiment, the adjustable fixation belt comprises a buckle and an adjusting ring. The adjusting ring is arranged on the adjustable fixation belt to adjust the length of the adjustable fixation belt. The buckle comprises a male part and a female part matched with the male part; the male part and the female part are arranged at two ends of the adjustable fixation belt respectively.

In a class of this embodiment, the first fastener is a hook-and-loop fastener.

In a class of this embodiment, the back piece comprises an upper edge at a position corresponding to a cranial part of the waist and a lower edge at a position corresponding to a caudal part of the waist. The middle portion of the upper edge protrudes upwards to form an arc-shaped edge; and the middle portion of the lower end edge to form an arc-shaped edge.

In a class of this embodiment, the back piece comprises two fabric layers; the two fabric layers are overlapped, and the cavity is formed between the two fabric layers.

In a class of this embodiment, the filling port is arranged on the upper edge or the lower edge.

In a class of this embodiment, the outer side face of the wing piece is provided with an auxiliary belt; the auxiliary belt is adapted to be held by the hand.

In a class of this embodiment, the filling port comprises a third fastener; the third fastener is a hook-and-loop fastener; and the third fastener is arranged on an opening of the filling port.

In a class of this embodiment, the second fastener is a hook-and-loop fastener.

According to the technical scheme, the waist fixation device is not only used for rigid fixation immediately post-fracture, but also for flexible fixation after surgery. The waist fixation device is shaped rapidly to match with the shape of the waist, and provides effective support to the lumbar vertebra. The waist fixation device provides a sufficient and appropriate stretching force, and is radiolucent. Therefore, the effect of maintaining fixation is good, the healing speed and quality are promoted, and the treatment and rehabilitation periods are shortened. The physiological characteristics of the spine are effectively restored, and the clinical complications of the patient are effectively reduced.

### BRIEF DESCRIPTION OF THE DRAWINGS

FIG. 1 illustrates a front view of a waist fixation device of the present disclosure;

FIG. 2 illustrates a rear view of a waist fixation device of the present disclosure; and

FIGS. 3A-3B illustrate views of the front and back surfaces of an auxiliary fixation belt of a waist fixation device.

### DETAILED DESCRIPTION OF THE EMBODIMENTS

For further illustrating the invention, experiments detailing a waist fixation device are described hereinbelow combined with the drawings. It should be noted that the following examples are intended to describe and not to limit the invention.

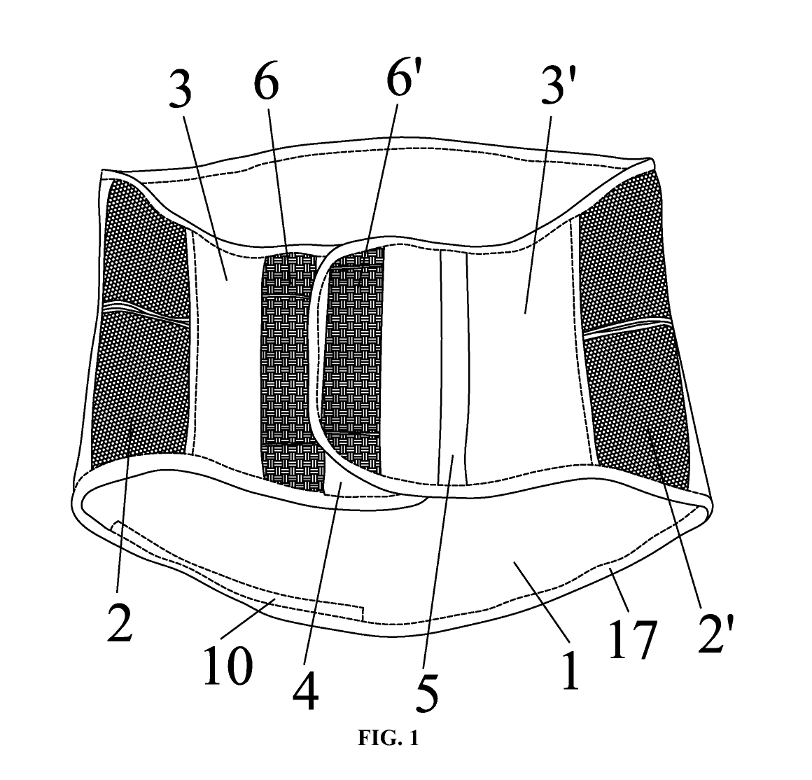

As shown in FIG. 1, the invention provides a waist fixation device, which comprises a back piece 1, a first elastic band 2, a second elastic band 2′, and a first fastener 4, wherein the first elastic band 2 and the second elastic band 2′ are arranged on the two sides of the back piece 1 respectively. When in use, the back piece 1 is attached to the posterior side of the waist of the patient, and the first elastic band 2 and the second elastic bands 2′ are respectively attached to both sides of the waist of the patient. Two pieces of the first fastener 4 are respectively arranged on two ends of the first elastic bands 2 and the second elastic band 2′. When two pieces of the first fastener 4 are connected, the entire waist fixation device wraps around the waist of the patient to support it. In some embodiments, the first fastener 4 is a hook-and-loop fastener.

A cavity is formed in the back waist piece, and a filling port 10 communicated with the cavity is arranged on the back piece 1. When in use, polyurethane slurry is injected into the cavity through the filling port 10, and then roll-homogenization treatment is performed on the polyurethane slurry on an operation table. The waist fixation device is wrapped around the waist of a patient, and after the polyurethane slurry enters the solid-phase softening section, the polyurethane slurry is solidified. The shape of the back piece 1 is matched with the posterior side of the waist of the patient. A rigid support is provided to the waist of the patient. Therefore, the attachment of the entire waist fixation device to the waist of the patient is improved, and the waist fixation device provides effective support to the lumbar vertebra. The polyurethane slurry is prepared by mixing special polyurethane slurry A and slurry B, and the specific technical scheme is disclosed in the U.S. Pat. No. 9,617,370. In some embodiments, a hook-and-loop fastener is arranged on an opening of the filling port 10 to facilitate closing or opening the filling port 10.

In some embodiments, the back piece 1 comprises an upper edge at a position corresponding to the upper part of the posterior side of the waist and a lower edge at a position corresponding to the lower part of the posterior side of the waist. The middle portion of the upper edge of the back piece 1 protrudes upwards to form an arc-shaped edge; the middle portion of the lower edge of the back piece 1 protrudes downwards to form an arc-shaped edge, so that the back piece can be better attached to the waist curve of the human body. In some embodiments, the filling port 10 is arranged on the upper edge or the lower edge.

In some embodiments, the cavity is formed by two fabric layers. Specifically, the two fabric layers are overlapped and combined, and the cavity is formed between the two fabric layers at their overlapping face. The two fabric layers are overlapped to form a covered edge 17.

In order to achieve a better fixing effect on the two sides of the waist of the patient, the first elastic band 2 and the second elastic band 2′ are respectively provided with the wing pieces 3, 3′, which are disposed on the two sides of the waist. The wing pieces 3, 3′ provide flexible support to the waist and fit with the shape of the waist of the patient. A sealing cavity 5 is formed in the wing pieces 3, 3′, and a supporting bar is placed in the sealing cavity 5, the supporting bars on the wing pieces 3, 3′ reinforce the flexion and control of the lumbosacral flexion side and protect the soft tissue of the lumbar sacrum. In some embodiments, the supporting bar has a herringbone shape, and comprises a main supporting bar in the middle and a plurality of auxiliary supporting bars extends from the main supporting bar to two sides for providing stable support. The sealing cavity 5 is made by two vertical sewing threads in the middle of the wing pieces 3, 3′.

In order to conveniently fasten the first fastener 4, the outer side faces of the wing pieces 3, 3′ are respectively provided with auxiliary belts 6, 6′. The auxiliary belts 6, 6′ are held by hand for pulling the wing pieces 3, 3′ together to fasten the two pieces of the first fastener 4.

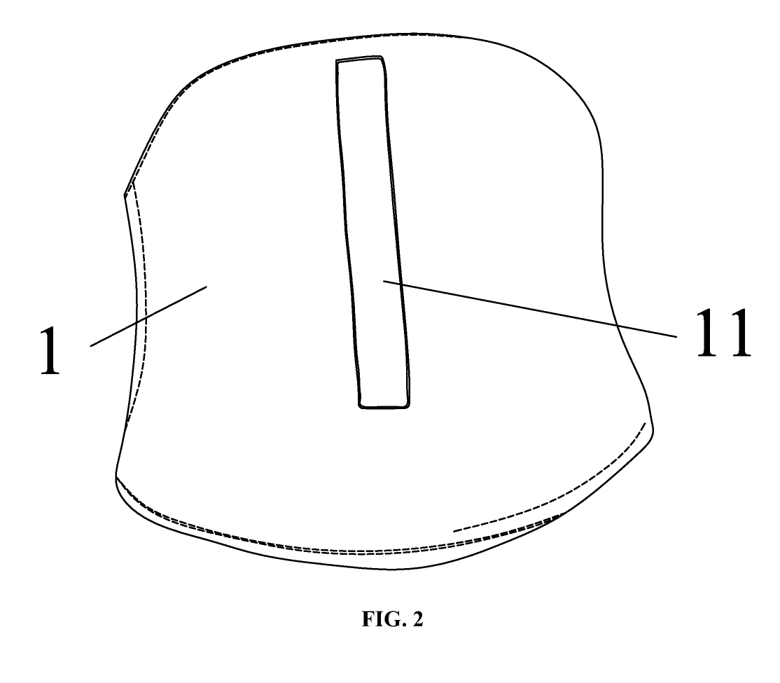

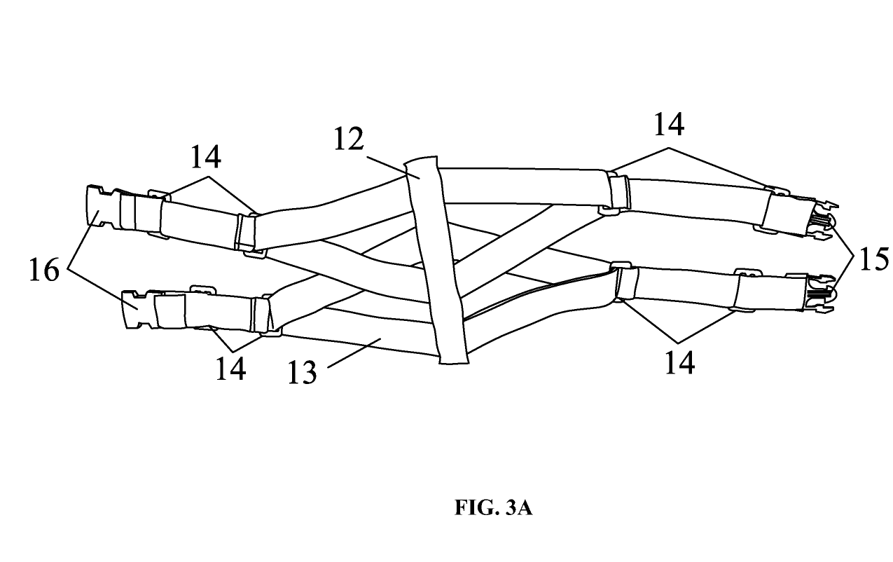

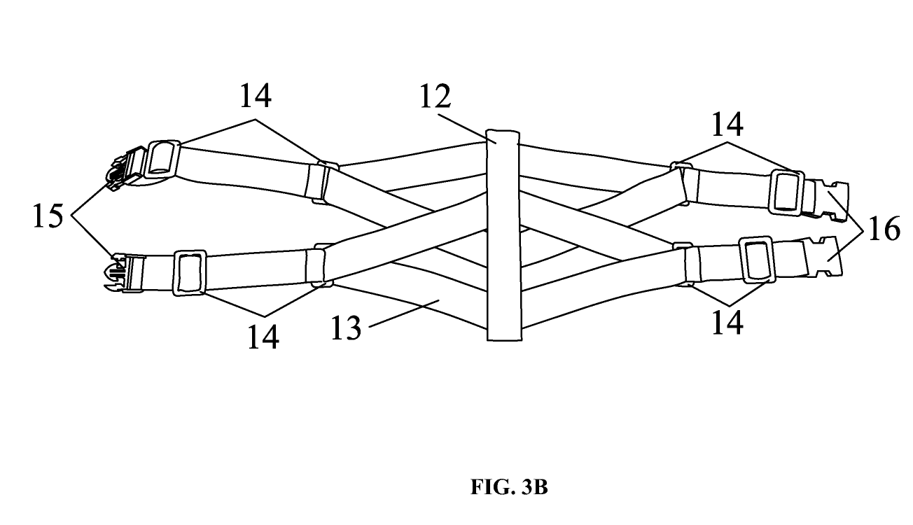

As shown in FIG. 2 and FIGS. 3A-3B, the waist fixation device comprises a buckle and an adjustable fixation belt 13, which are adapted to adjust the length of the fixation belt 13, and the adjustable fixation belt 13 is connected to the back piece 1 through a second fastener. In some embodiments, the second fastener is a hook-and-loop fastener. Specifically, a first piece 11 of the second fastener is arranged in the middle of the outer side of the back piece, and a second piece 12 of the second fastener is arranged in the middle of the adjustable fixation belt. When in use, the adjustable fixation belt 13 is fixedly connected with the back piece 1, and then the male part 15 of the buckle and the female part 16 of the buckle at the two ends of the adjustable fixation belt are buckled, and finally the adjusting ring 14 on the adjustable fixation belt 13 is adjusted to achieve a desired length of the adjustable fixation belt 13.

The waist fixation device comprises a rigid fixing part and a flexible fixing part to respectively provide rigid support and flexible support to the waist, which is different from the elastic bandage. The waist fixation device is convenient to use, can be shaped rapidly with the body and has a sufficient and appropriate stretching force, and is radiolucent. The fixation effect is good, the treatment requirements of patients can be fully met, and the treatment and rehabilitation periods are shortened.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A fixation device for waist, comprising:
a) a back piece; the back piece comprising a cavity and a filling port;
b) a first elastic band; the first elastic band comprising a first end;
c) a second elastic band; the second elastic band comprising a second end;
d) a first fastener; and
e) two wing pieces; each of the two wing pieces comprising a sealing cavity and a supporting bar;
wherein:
the back piece is adapted to cover a posterior side of the waist;
the first elastic band and the second elastic band are respectively connected to two sides of the back waist piece;
a first piece of the first fastener is disposed on the first end and a second piece of the first fastener is disposed on the second end to connect the first end and the second end, for keeping the fixation device wrapped around the waist;
the cavity is arranged in the back piece;
the filling port is arranged on the back piece and is communicated with the cavity;
when in use, polyurethane slurry is injected into the cavity through the filling port, after the polyurethane slurry enters a solid-phase softening section, a shape of the back piece is matched with a shape of the posterior side of the waist;
the two wing pieces are respectively disposed on outer sides of the first elastic band and the second elastic band;
the sealing cavity is arranged in the wing piece; and
the supporting bar is placed in the sealing cavity.

2. The device of claim 1, wherein:
the fixation device further comprises an adjustable fixation belt and a second fastener;
the adjustable fixation belt is adapted to fix the fixation device on the waist; and
a first piece of the second fastener is disposed on the adjustable fixation belts and a second piece of the second fastener is disposed on the back piece for fixing the adjustable fixation belt on the back piece.

3. The device of claim 2, wherein:
the adjustable fixation belt comprises a buckle and an adjusting ring;
the adjusting ring is arranged on the adjustable fixation belt to adjust the length of the adjustable fixation belt;
the buckle comprises a male part and a female part matched with the male part; and
the male part and the female part are arranged at two ends of the adjustable fixation belt respectively.

4. The device of claim 1, wherein the first fastener is a hook-and-loop fastener.

5. The device of claim 1, wherein:
the back piece comprises an upper edge and a lower edge;
the upper edge is at a position corresponding to an upper part of the posterior side of the waist;
the lower edge is at a position corresponding to a lower part of the posterior side of the waist;
a middle portion of the upper edge protrudes upwards to form an arc-shaped edge; and
a middle portion of the lower edge protrudes downwards to form an arc-shaped edge.

6. The device of claim 2, wherein:
the back waist piece comprises two fabric layers;
the two fabric layers are overlapped; and
the cavity is formed between the two fabric layers.

7. The device of claim 6, wherein the filling port is arranged on the upper edge or the lower edge.

8. The device of claim 7, wherein
an outer side face of each of the two wing pieces is provided with an auxiliary belt; and
the auxiliary belt is adapted to be held by hand.

9. The device of claim 8, wherein
the filling port comprises a third fastener;
the third fastener is a hook-and-loop fastener; and
the third fastener is arranged on an opening of the filling port.

10. The device of claim 9, wherein the second fastener is a hook-and-loop fastener.
